(19) United States
(12) Patent Application Publication
(10) Pub. No.: US 2013/0006246 A1
(43) Pub. Date: Jan. 3, 2013

(54) Distal Radius Plate

(76) Inventor: Mark Dodson, Alexandria, LA (US)

(21) Appl. No.: 13/174,429

(22) Filed: Jun. 30, 2011

(51) Int. Cl. A61B 17/80

(52) U.S. Cl. 606/70

(57) ABSTRACT

A distal radius plate for repairing distal radius fractures is provided, comprising a proximal member and a distal member, wherein the proximal member includes a pair of proximal screw holes, a pair of distal screw holes; an elongated slot located between the proximal screw holes and the distal screw holes, adapted to allow placement of one or more screws within the slot; and a notch formed into the terminal end of the proximal member adapted to receive a retractor tip. The distal member is inclined with respect to the proximal member, and includes a plurality of holes for pegs and K-wires.

### CROSS-REFERENCE TO RELATED APPLICATIONS

Not applicable.

### STATEMENT REGARDING FEDERALLY SPONSORED RESEARCH OR DEVELOPMENT

Not applicable.

### THE NAMES OF THE PARTIES TO A JOINT RESEARCH AGREEMENT

Not applicable.

### INCORPORATION-BY-REFERENCE OF MATERIAL SUBMITTED ON COMPACT DISC

Not applicable.

### BACKGROUND OF THE INVENTION

### Field of the Invention

The present invention relates to devices and methods used to repair bone fractures, and more particularly to such devices used to repair distal radius fractures on the palmar or volar side.

### BRIEF DESCRIPTION OF THE DRAWINGS

For a further understanding of the nature, objects, and advantages of the present invention, reference should be had to the following detailed description, read in conjunction with the following drawings, wherein like reference numerals denote like elements.

FIG. 1A shows a top view of a distal radius plate in accordance with the present invention.

FIG. 1B shows an end view of the embodiment of FIG. 1, depicting a notch for a Hohmann retractor.

FIG. 2 shows a side view of the embodiment of FIG. 1.

### DETAILED DESCRIPTION OF THE INVENTION

Before the subject invention is further described, it is to be understood that the invention is not limited to the particular embodiments of the invention described below, as variations of the particular embodiments may be made and still fall within the scope of the appended claims. It is also to be understood that the terminology employed is for the purpose of describing particular embodiments, and is not intended to be limiting. Instead, the scope of the present invention will be established by the appended claims.

In this specification and the appended claims, the singular forms “a,” “an,” and “the” include plural reference unless the context clearly dictates otherwise. Unless defined otherwise, all technical and scientific terms used herein have the same meaning as commonly understood to one of ordinary skill in the art to which this invention belongs.

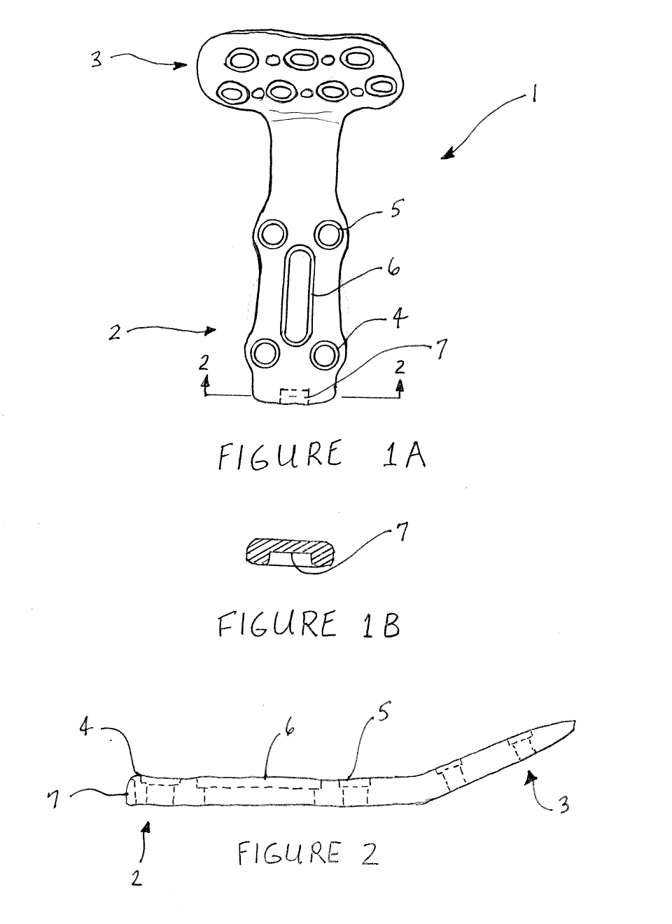

Turning now to the figures, FIGS. 1A and 1B show top and proximal end views of a preferred embodiment of the distal radius plate 1 of the present invention. The plate 1 includes a proximal member 2 and a distal member 3. The proximal member 2 of the plate 1 includes a pair of proximal screw holes 4 (radius and ulnar), a pair of distal screw holes 5 (radius and ulnar), and an elongated slot 6 for one or two additional screws. Therefore, the improved plate 1 allows for as many as six screws to be placed into the bone within a more confined area, compared to three screws in most other distal radius plate designs.

The proximal screw holes 4 are formed approximately 20 mm-25 mm apart from one another, although the actual distance may be modified to suit the needs of the surgical situation. Similarly, the distal screw holes 5 are formed approximately 20 mm-25 mm apart from one another, and subject to modification as explained previously. The distance between the proximal screw holes 4 and the distal screw holes 5 is approximately 40 mm-50 mm, although such distance can be modified for predetermined plate sizes for different surgical applications. The elongated slot 6 is approximately 25 mm long, thus accommodating at least two cortical screws for greater fixation stability. The use of an increased number of screws increases the fixation points and distributes the forces over a wider area. This technique is believed to increase the strength of the repair, which is particularly important in osteoporotic bone.

The screws can be placed in a locked or unlocked configuration. The screws can be angled or the traditional perpendicular position (90 degrees). Angled screws will increase the length of the construct without increasing the need for a longer incision.

The shorter plate design of the present invention decreases the incision length and the amount of muscle stripping from the volar (palmar) side of the radius. Reducing the length of the incision may reduce blood loss, decrease swelling, and improve early and late return of motion.

Also included is a small notch or recess 7 formed into the proximal member 2, as shown in FIGS. 1A and 1B. The notch 7 allows for the convenient placement of the tip of a Hohmann retractor. In prior designs, there is no easy way to place a retractor at the end of the plate 1 to move soft tissue away from the incision. When the retractor is placed into the notch 7, skin and other soft tissue are easily moved away from the plate 1, because the notch 7 provides the necessary leverage point.

As can be seen more clearly in FIG. 2, the plate 1 has a generally familiar shape typical of other known distal radius plate designs. However, the placement of the proximal and distal holes 4, 5, and the elongated slot 6 provide substantially greater flexibility in screw placement, depending on the needs of the repair.

All references cited in this specification are herein incorporated by reference as though each reference was specifically and individually indicated to be incorporated by reference. The citation of any reference is for its disclosure prior to the filing date and should not be construed as an admission that the present invention is not entitled to antedate such reference by virtue of prior invention.

It will be understood that each of the elements described above, or two or more together may also find a useful application in other types of methods differing from the type described above. Without further analysis, the foregoing will so fully reveal the gist of the present invention that others can, by applying current knowledge, readily adapt it for various applications without omitting features that, from the standpoint of prior art, fairly constitute essential characteristics of the generic or specific aspects of this invention set forth in the appended claims. The foregoing embodiments are presented by way of example only; the scope of the present invention is to be limited only by the following claims.

The invention claimed is:

1. A distal radius plate for repairing distal radius fractures, comprising:
a proximal member and a distal member, wherein the proximal member includes:
(a) a pair of proximal screw holes;
(b) a pair of distal screw holes;
(c) an elongated slot adapted to allow placement of one or more screws; and
(d) a notch formed into the terminal end of the proximal member adapted to receive a retractor tip; and
wherein the distal member includes a plurality of holes.
